(19) United States
(12) Patent Application Publication
(10) Pub. No.: US 2014/0107501 A1
(43) Pub. Date: Apr. 17, 2014

(54) WIRELESS MULTI-SENSOR PLATFORM FOR CONTINUOUS REAL-TIME MONITORING OF CARDIOVASCULAR RESPIRATORY DYNAMICS

(71) Applicant: THE BOARD OF REGENTS FOR OKLAHOMA STATE UNIVERSITY, STILLWATER, OK (US)

(72) Inventors: Ranga Komanduri, Stillwater, OK (US); Satish T.S. Bukkapatnam, Stillwater, OK (US); Trung Le, Stillwater, OK (US)

(73) Assignee: THE BOARD OF REGENTS FOR OKLAHOMA STATE UNIVERSITY, STILLWATER, OK (US)

(21) Appl. No.: 14/022,004

(22) Filed: Sep. 9, 2013

Related U.S. Application Data
(60) Provisional application No. 61/698,117, filed on Sep. 7, 2012.

(51) Int. Cl. A61B 5/04; A61B 5/0205

(52) U.S. Cl. CPC A61B 5/04011; A61B 5/0205
USPC 600/484; 600/509; 600/523; 600/512

(57) ABSTRACT

A set of vectorcardiogram leads attach to a patient and a signal conditioning circuit receives a plurality of analog signals from the plurality of vectorcardiogram leads. An analog to digital converter that transforms the conditioned analog signals into digital signals and a processor transforms the digital vectorcardiogram signals into electrocardiogram signals.

### CROSS REFERENCE TO RELATED APPLICATION

This application claims the benefit of U.S. Provisional Application No. 61/698,117 filed Sep. 7, 2012, herein incorporated by reference in its entirety for all purposes.

### STATEMENT REGARDING FEDERALLY SPONSORED RESEARCH OR DEVELOPMENT

This invention was made with government support under Grant No. CMMI-0729552 awarded by the National Science Foundation. The government has certain rights in the invention.

### FIELD OF THE INVENTION

This disclosure is related to electrocardiogram signals in general and, more particularly, to a system providing continuous, real-time, wireless monitoring of electrocardiogram signals.

### BACKGROUND OF THE INVENTION

Cardiac disorders are the leading cause of mortality in the world. While considerable advances have taken place in the clinical diagnosis of various cardiac disorders, prognostic approaches vital for preventive interventions have not received the same attention. Current approaches mostly focus on deriving general outlooks based on group behaviors and some simple cardiac signal trends. Rich information discernible from longitudinal tracking of physiological signals, such as ECG, heart sound, and pulse, remains relatively unexplored.

Early, individualized prognosis, especially for at-risk (e.g., critical care) populations, and those subjected to intense physical activity (e.g., sports training) can significantly reduce mortality and morbidity risks.

What is needed is a system for addressing the above, and related, concerns.

### SUMMARY OF THE INVENTION

The invention of the present disclosure, in one aspect thereof, comprises a system having a plurality of electrical detector leads for attaching to a patient. The system has a signal conditioning circuit that receives a plurality of signals from the detector leads and applies amplification, and high and low pass filtering to the received signals to produce conditioned analog signals. The system also has an analog to digital converter than converts the conditioned analog signals to digital signals, and a microprocessor that applies a transformation to the digital signals to produce a set of transformed signals that are greater in number than the plurality of analog signals. A display device that selectively displays the digital signals and the transformed signals.

In some embodiments, the system includes a memory associated with the microprocessor for storing the digital signals. A wireless communication chip may be utilized to transmit the digital signals to a recipient device.

In some embodiments, the microprocessor computes heart rate variability for display on the display device. The plurality of detector leads may correspond to a 3-lead vectorcardiogram. The digital signals correspond to a 12-lead electrocardiogram. The microprocessor may compute heart rate variability based on an R-R timing obtained from the electrocardiogram digital signals. The microprocessor may also calculate energies of 3 separate vectorcardiogram leads for display on the display device. The system may have a microphone that captures heart sounds, wherein the microprocessor correlates the electrocardiogram signals to the heart sounds to determine respiration events.

The invention of the present disclosure, in another aspect thereof, comprises a system having a set of vectorcardiogram leads for attaching to a patient, a signal conditioning circuit that receives a plurality of analog signals from the plurality of vectorcardiogram leads, an analog to digital converter that transforms the conditioned analog signals into digital signals, and a processor that transforms the vectorcardiogram signals into electrocardiogram signals.

The signal conditioning circuit and the processor may be packaged into a portable electronic device. The portable electronic device may further comprise a display that selectively displays the vectorcardiogram and the electrocardiogram. In some embodiments, the vectorcardiogram is a 3 lead vectorcardiogram and the electrocardiogram is a 12 lead electrocardiogram. The microprocessor may perform an affine transform of the 3 lead vectorcardiogram to produce the 12 lead electrocardiogram.

The system may further comprise a memory that stores the digital signals and may have a wireless transmitter that delivers at least the 3 lead vectorcardiogram to another device. In some embodiments, the system has a microphone for detecting heart sounds and the processor correlates the heart sounds to the ECG signal

The invention of the present disclosure, in another aspect thereof, comprises a method that includes attaching a portable device to a patient that obtains 3 lead vectorcardiogram reading from a patient, transforming the vectorcardiogram in the portable device to a 12 lead electrocardiogram, and displaying the 12 lead electrocardiogram.

The method may include transforming the vectorcardiogram in the portable device to a 12 lead electrocardiogram by performing an affine transform. The 12 lead electrocardiogram may be displayed and the vectorcardiogram wirelessly transmitted.

### BRIEF DESCRIPTION OF THE DRAWINGS

FIG. 1 is a high level view of a wireless heart monitoring system according to the present disclosure.

FIG. 2 is a perspective view of the system of FIG. 1 deployed on the torso of a patient.

FIG. 3 is a schematic diagram of a power supply for a wireless heart monitoring system.

FIG. 4 is a schematic diagram of an analog signal conditioning circuit for a wireless heart monitoring system.

FIG. 5 is a schematic view of a printed circuit board implementing a portion of the wireless heart monitoring system of the present disclosure.

FIG. 6 is a perspective of a Bluetooth® transmission module.

FIG. 7 is an exemplary vectorcardiogram.

FIG. 8 is a heat model with integrated vectorcardiogram.

FIG. 9 is an illustration of data gathered and displayed by the wireless heart monitoring system of the present disclosure.

FIG. 10 is another illustration of data gathered and displayed by the wireless heart monitoring system of the present disclosure.

FIG. 11 is a functional component diagram of a cardiac monitoring system according to the present disclosure.

FIG. 12 illustrates a correlation between heart sound signals and VCG signals.

### DETAILED DESCRIPTION OF THE PREFERRED EMBODIMENTS

Recent developments in wireless communication, including miniaturized sensors using micro-electro-mechanical (MEMS) or nano-electro-mechanical (NEMS) technology, and nonlinear physiological signal analysis offer a unique opportunity to enhance continuous quantitative prognosis of cardiac disorders of these populations without undue curtailment of their mobility. Lately, the use of wireless technologies for critical care has evoked significant interest. But harnessing of the complex spatio-temporal patterns of these wireless signals, so vital for effective prognosis, has not received due attention.

In various embodiments, the present disclosure describes a real time wireless electrocardiogram (ECG) monitoring system. The system may utilize wireless Bluetooth® technology for data communication. The wireless ECG monitoring system is suitable for continuous real time monitoring. The system may be used in at least two configurations. These include measuring 12-lead ECG and/or 3-lead vectorcardiogram (VCG). The leads are connected to a lightweight, portable module, which can be easily carried along with the person. The module transmits the signal wirelessly to the computer or a portable device such as, an iPhone or Windows mobile phone, or Google Android phone. The signal may be displayed in real time on a portable handheld device and stored on a remote server for telemedicine applications.

The technology of the present disclosure is more energy efficient (longer battery life), has longer transmission range (improved mobility), and higher sampling rates (improved diagnostic power under multisensory regime) than prior systems. In addition to the traditional feature extraction techniques, the disclosed system tracks advanced VCG based features to improve the diagnostic accuracy of the computer based algorithms.

In one embodiment, with a 9V battery, the present system provides a 72-hour run time. It has a transmission range of 100 m relying on Class I Bluetooth® protocol. It samples at a resolution of 16 bits at a frequency of 250-500 Hz per channel. Frequency response is 0.176-90 Hz. The device provides up to 7 leads and 3 display channels. The device also has the ability to produce vectorcardiograms (VCG). All this can be integrated into a small form factor of about 50×30 mm.

Although some embodiments utilize Bluetooth® it is understood that other communications protocols may be utilized instead or additionally. Other protocols may include Zigbee, Wi-Fi, CDMA (Code division multiple access), 3G network, and possibly wired communication module. The devices of the present disclosure meet all applicable regulations regarding the use of electricity. They are FDA compliant and are compatible with various wireless network security protocols. Encryption may be employed to ensure compliance with HIPAA (Health Insurance Portability and Accountability Act) and other applicable regulations and standards.

Various embodiments of the present disclosure may be utilized with iPhone, windows mobile, Linux android platform, and other hardware. A single device may be capable of interfacing with a variety of devices of various operating systems. In some embodiments, the underlying software is written using MatLab, a multiple platform program. Hence, the device software can be used in Linux, Windows, and Macintosh environments with MatLab installed. In one embodiment the device transmits data wirelessly to the computer and MatLab for displaying and saving ECG data.

In embodiments that interface with mobile phones or other portable devices, real-time transmission of the collected data over the cellular network may be provided. This information may be sent to servers, remote data storage, and/or to physicians or other health care providers for review. An on board SD flash memory card may be included on the device to save data locally. Some embodiments include a screen and operate without the need of a laptop or mobile phone to record data.

The system may use the Bluetooth® Class I transmission protocol. The maximum data transmission operation range is up to (333 inches) 100 m. The compact design of the Bluetooth module allows the whole system is integrated into a small and compact unit which is very mobile and compatible. Furthermore, the advantages of easy connectivity feature of blue-tooth protocol enable the system to connect with other blue-tooth platform devices. Therefore, the system can expand the ability to connect with other wireless medical system through peer to peer or client server connection. In addition, the ability to connect using Bluetooth protocol enables the system to connect with a remote server using the mobile devices and networks that support the Bluetooth platform.

In some embodiments, the system utilizes (MEMS) based sensors for the measurement of bio-electrical signals. The small size of MEMS based sensors make them particularly useful for the ECG measurement in small children and infants. In addition to ECG sensors, a multi-sensor embodiment is contemplated that combines sensor input from acoustic sensors to record sound signals and one or more accelerometers to record vibration signals. The signals may be collected simultaneously. The combined information is used to reduce or eliminate artifacts in the ECG signal caused by muscle movement, and also to provide additional features to improve the diagnostic accuracy.

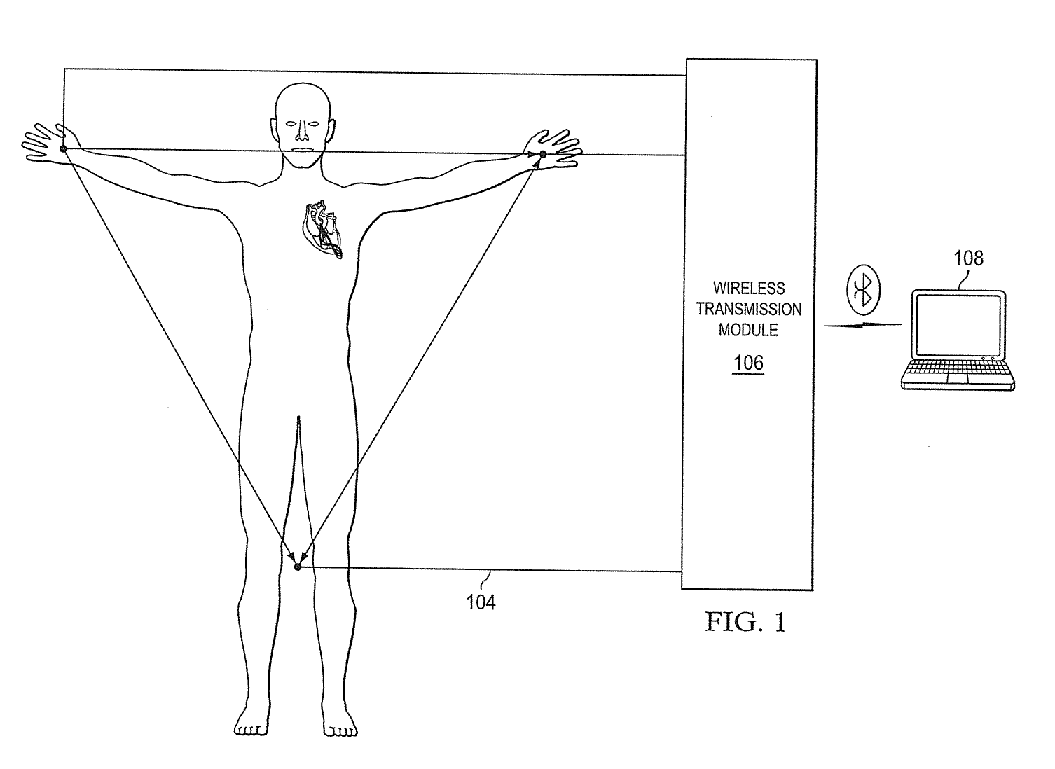

A high level conceptual view of an exemplary monitoring system 100 according to the present disclosure attached to a patient is shown in FIG. 1. Here the patient 102 is fitted with a number of ECG leads 104 as is known in the art. The leads 104 are electrically connected to a wireless transmission module 106 that provides voltage signals obtained via the leads 104 to a mobile device 108. In the present case, the mobile device 108 is a laptop. It will be appreciated that other mobile devices may be suitable, including tablets or smartphones.

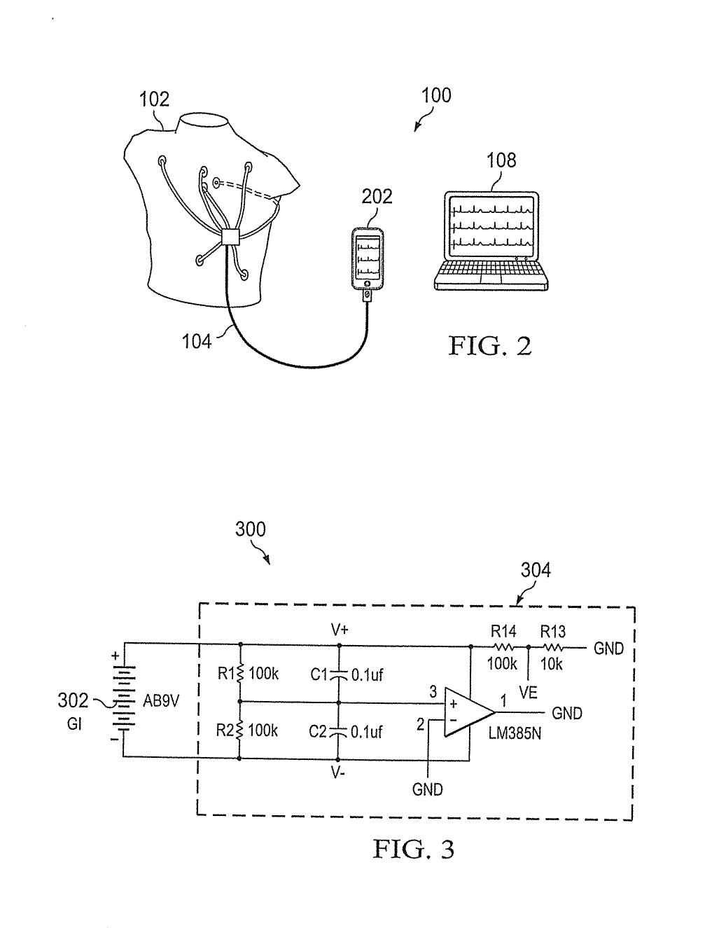

FIG. 2 provides a more detailed view of the manner in which the leads 104 may attach to a torso of the patient 102. Here again, the manner of attaching ECG leads is known to those of skill in the art. Furthermore, the present disclosure is not meant to be limited by the number of leads utilized (e.g., 7 leads, 12 leads, or another number). Furthermore, some leads may be acoustic sensor (e.g., hear sound leads). Here the leads 104 attached to a portable device such as mobile phone 202. The phone 202 may be a smart phone capable of reading voltages provided by the leads 104 and producing a graphic display of the ECG signals. In some embodiments, the phone 202 transmits the ECG signals wirelessly (e.g., via Bluetooth) to a computer 108 or other device. The computer 108 is not necessarily a laptop or portable computer. For example, it may be a stationary or desktop computer with a Bluetooth module. In some embodiments, the phone 202 will transmit the signals collected in real time to the computer 108. It may display signals corresponding to the collected ECG signals simultaneously. In some embodiments, the collected signals are stored in the internal memory of the phone 202 in addition to, or instead of, being transmitted to the computer 108. The phone 202 may also be programmed to transmit collected ECG signals after they have been collected. In such case, the phone 202 replaces a dedicated wearable ECG or other data collection unit.

In some embodiments, where standalone data collection module is utilized, a 9V power supply 300 can be used (as shown in FIG. 3). In some embodiments the power supply 300 may utilize a commercially available 9V battery 302. The full circuitry is shown in FIG. 3, including signal conditioning circuitry 304. However, it is understood that the power supply 300 is only exemplary, as many other configurations are possible for one skilled in the art.

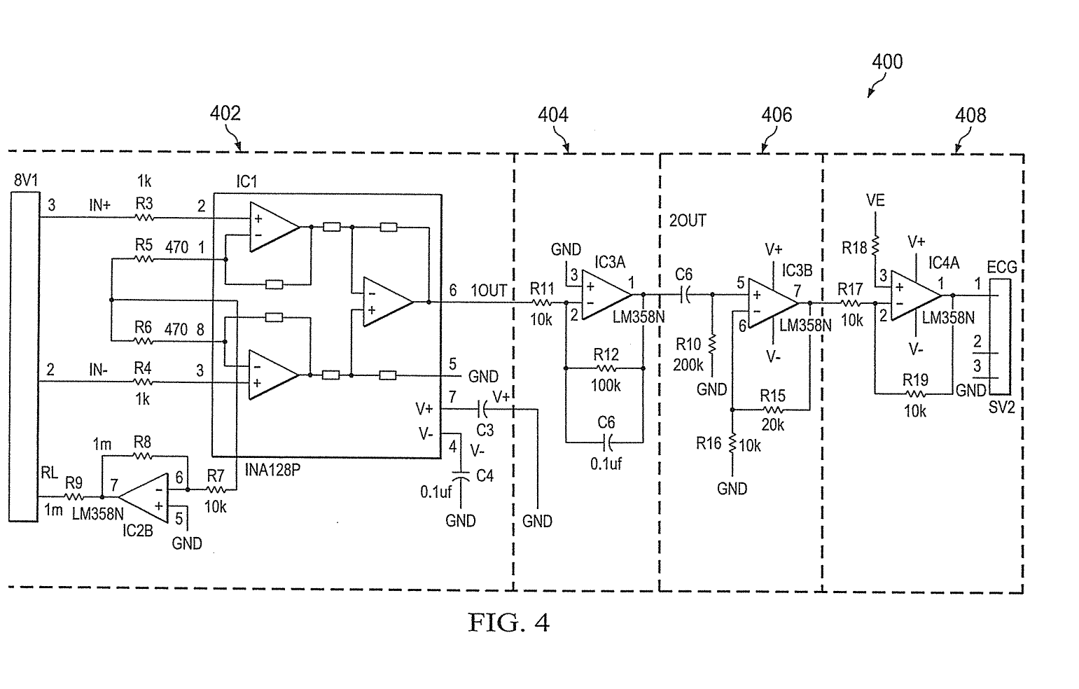

Referring now to FIG. 4, an analog front end amplifier (AFE) 400 suitable for use with the devices of the present disclosure is shown. In the present embodiment, the AFE 400 performs the necessary signal conditioning of the obtained analog signal(s) to enable them to be properly read and displayed for review by a medical professional. Incoming signals may first be amplified by a differential amplifier circuit 402. A low pass filter circuit 404 may be applied to the amplified signals. In some embodiment, this circuit 404 suppresses signals above about 50 Hz. A high pass filter 406 may also be applied to suppress signals below about 0.5 Hz. These serve to mitigate baseline wandering and signal drift in the amplified signals. Appropriate offset and inversion operations may be applied to the resulting signal by the circuit 408. Here again, the circuitry of FIG. 4 is shown is for purposes of illustration only and the devices of the present disclosure are not meant to be limited to this particular circuitry. One of skill in the art will recognize that the required functionality may be implemented in various ways. In some embodiments, all or a portion of the signal conditioning could be handled digitally (e.g., using a DSP chip that is part of a standalone unit, or inside the smart phone 202 or portable device 108).

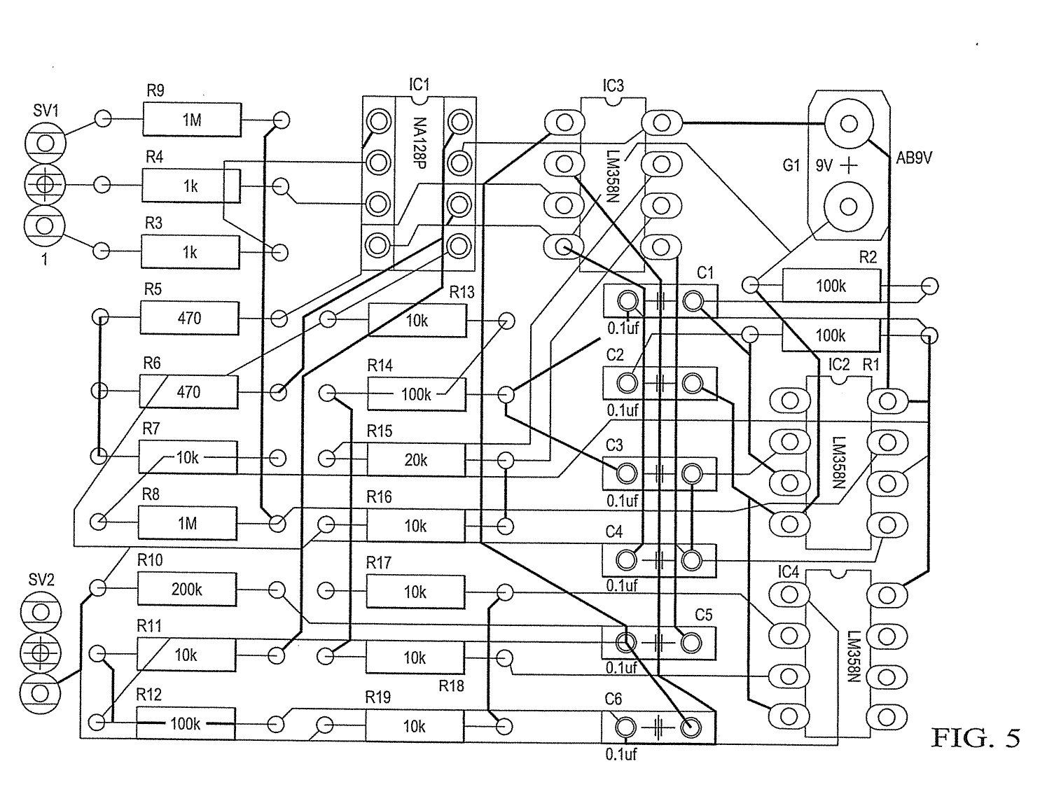

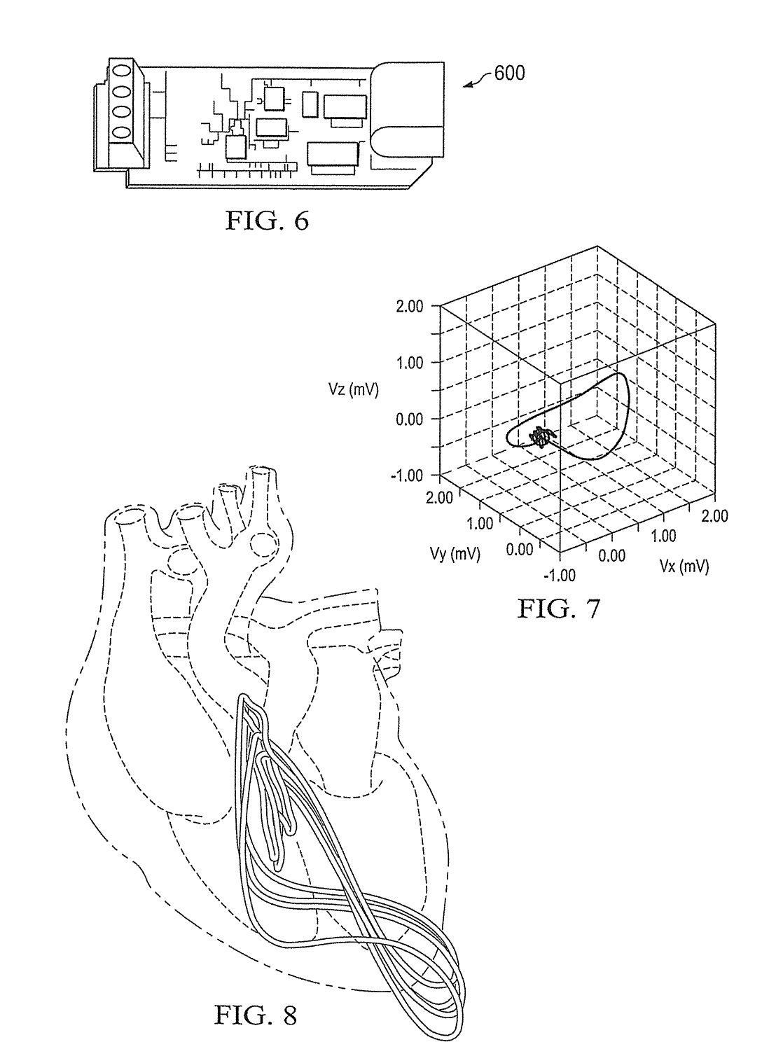

The AFE 400 and other components may be located on a printed circuit board (PCB) such as the one shown diagrammatically in FIG. 5. The PCB of FIG. 5 can gather 3-channel Frank X, Y Z VCG signals plus two channels for sound and vibrations at high resolution for further digitization, processing, and transmission. The PCB of FIG. 5 is illustrative only, as many other configurations are possible.

Once the signals are acquired in analog, they may be converted to digital form and transmitted wirelessly. The A/D conversion can be handled by the phone 202 or a dedicated standalone A/D unit. In cases where the Bluetooth® protocol is utilized, the BlueSentry data acquisition and control module available from Ad Hoc Electronics (http://www.adhocelectronics.com) may be utilized. The BlueSentry device contains an 8 channel 16 bit analog to digital AD converter. The device can sample analog signals and converting them to a Bluetooth-enabled digital data stream. The device also has two general purpose digital in/outputs and 2 field effect transistor (FET) power switches for controlling sensor power that can be configured over the same Bluetooth link using simple output commands. The BlueSentry device can be connected using Serial Port Profile directly to Bluetooth clients, giving computers, PDAs, smartphones, or tablets the ability to directly obtain data from the sensors.

In some embodiment, rather than using up to 12 electrodes to measure a standard 12-lead ECG, six electrodes can be used (excluding a ground electrode integrated into the device band). These electrodes create three orthogonal leads that measure the heart's activity in the X, Y and Z directions, (also known as a vectorcardiogram or VCG). Vectorcardiography depicts spatial electrical heart activity. An additional alternate configuration uses two electrodes that produce a single lead. By using fewer leads, the device 100 is capable of real time display and high resolution ECG data wireless transmission via Bluetooth. The device software displays the vectorcardiogram in real time in addition to the Frank leads. An exemplary VCG is shown in FIG. 7.

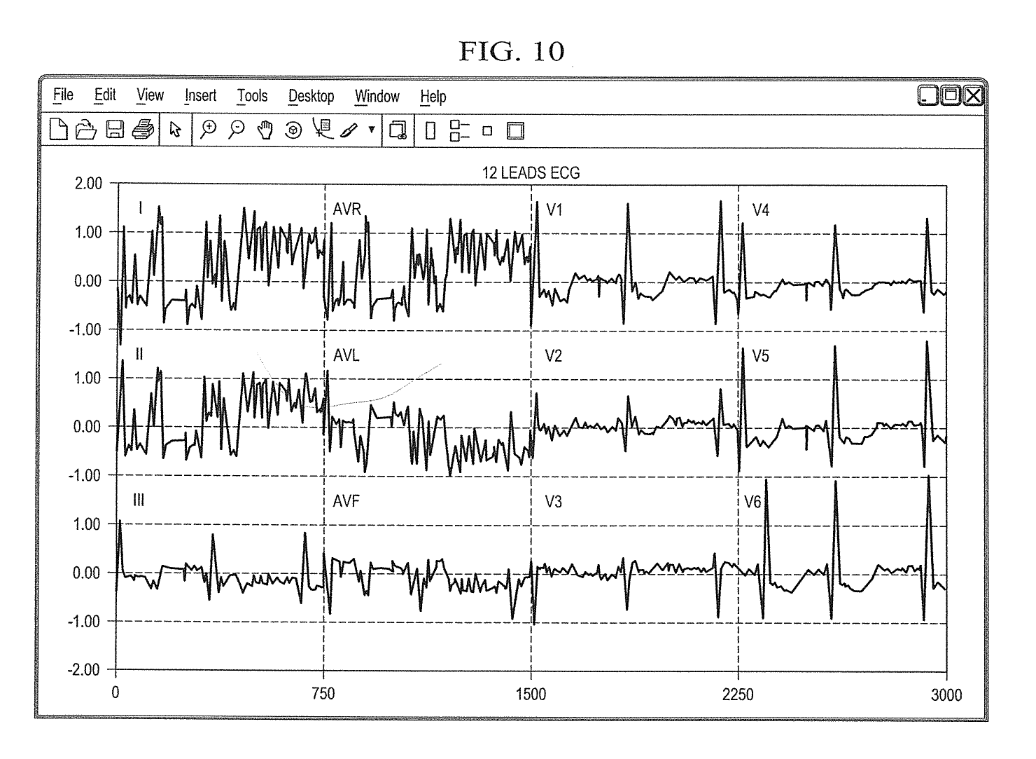

The VCG data can be utilized along with anatomical three-dimensional heart models as shown in FIG. 8. Real time feature extraction allows for signal segment separation. Each portion of the ECG waveform may be colored specifically to show QRS, P and T waves. An additional feature that may be implemented in hardware and/or software uses a transformation matrix to transform the measured Frank Leads into a standard 12-lead ECG, which is the primary diagnosis test used by physicians. As explained more fully below, a linear transformation matrix reduces processing power necessary to display 12-lead ECG in real time. Data storage requirements are greatly reduced by saving recordings as only three leads rather than standard 12-leads. The linear affine transform enables lossless conversion to 12-lead nullifying the necessity to save 12-lead signals in addition. FIG. 10 illustrates a standard ECG display providing a 12-lead signal obtained using the measured VCG as described above.

In addition to record and/or displaying data acquired, the system of the present disclosure provides for feature extraction capabilities. This important tool allows a healthcare provider to focus on the most important parts of the acquired data.

As implemented in the hardware and/or software of the present disclosure, data relating to heart rate can be extracted from the signals obtained from the patient. Heart variability is available on a real-time basis. R peaks are detected as the ECG signal is recording, and the heart rate is calculated based on the time interval between two consecutive R peaks. In addition to heart rate, the energy of the individual leads may be detected and provided. The energy of the individual leads (Vx, Vy, Vz) in the frequency bandwidth from 0-40 Hz may be measured. Energy is calculated by the summation of square amplitude of total frequency components. Energy may be calculated and displayed in real-time in separate Vx, Vy, Vz modes. This calculated and displayed energy can reveal sudden changes in the individual leads to separate the origin of the change corresponding axis of the heart. Based on the change in the heart energy, cardiologists or doctors can trace problems and respond properly.

Various embodiments can also calculate frequency spectrum. This provides the spectrum of combination of three leads VCG in the bandwidth 0-40 Hz in frequency domain. Fourier transformation may be applied to the recorded ECG signals. Coefficients of the corresponding frequency are displayed in the real-time. Furthermore, these coefficients are used to calculate the energy of the individual leads.

It will be appreciated that, besides heart rate, there are many other frequency components that appear in the frequency spectrum. Real-time frequency spectrum provides another channel of information that doctors can utilize for diagnostic or treatment purposes.

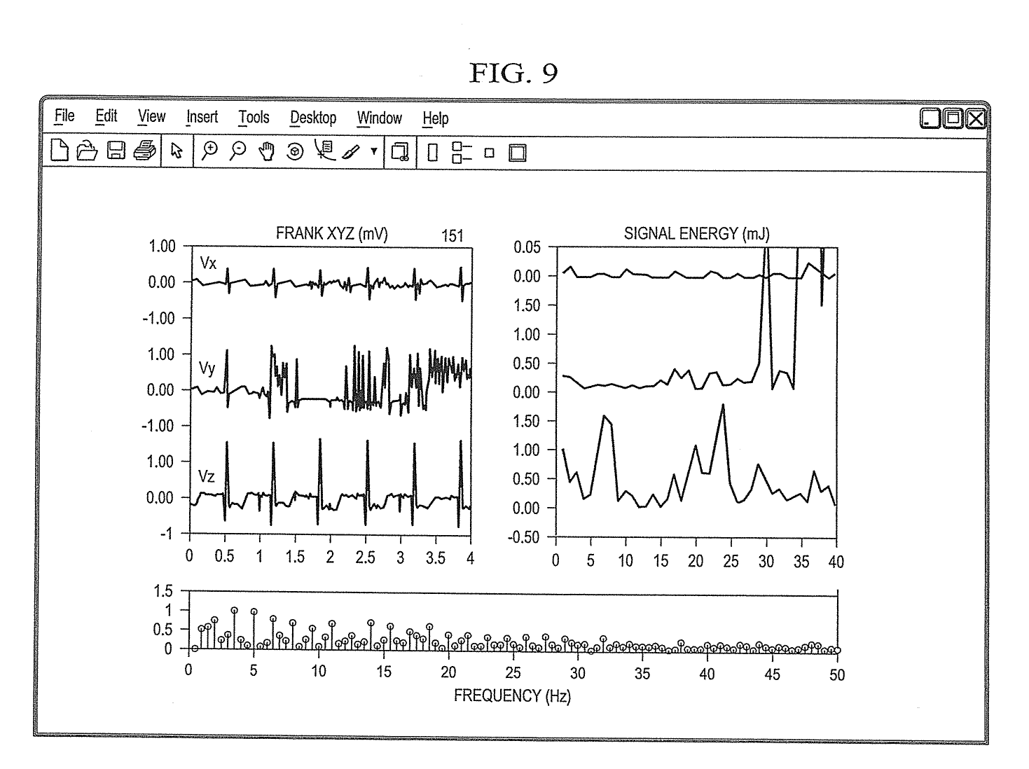

FIG. 9 provides an example of how the aforedescribed information may be presented to a user. FIG. 9 is a display suitable for use with a computer monitor. However, space permitting, this information may be displayed on a portable device. In some embodiments, only a portion of it may be displayed at one time. The systems of the present disclosure include software routines and suites (implemented in hardware, software, or a combination of the two) that visualize the digital signals obtained from a wearable device (e.g., for display as shown in FIGS. 7-10). The software may provide data as filtered through a plurality of filters including VCG, octant, conventional 12-lead, and frequency spectrum, etc.

In addition to the foregoing, the systems of the present disclosure, in various embodiments are able to provide further identification of critical points within the collected data. For example, VCG-critical events including P, Q, R, S, T are extracted in real-time. R peaks may be detected and a phase space method applied to detect the other events: P, Q, S, T. All of the critical points are extracted as soon as the signal is recorded by the system. Critical points are used in the software to calculate the heart-rate. P, Q, R, S, T may be used to display time dependent 3-D VCG loops. The display of 3-D VCG in the real-time with the annotations of P, Q, R, S, T in each loop provide spatial and temporal view of the heart axis. This information is suitable to keep track change in VCG loops. Coherence among VCG and sound signals from the patient may also be monitored.

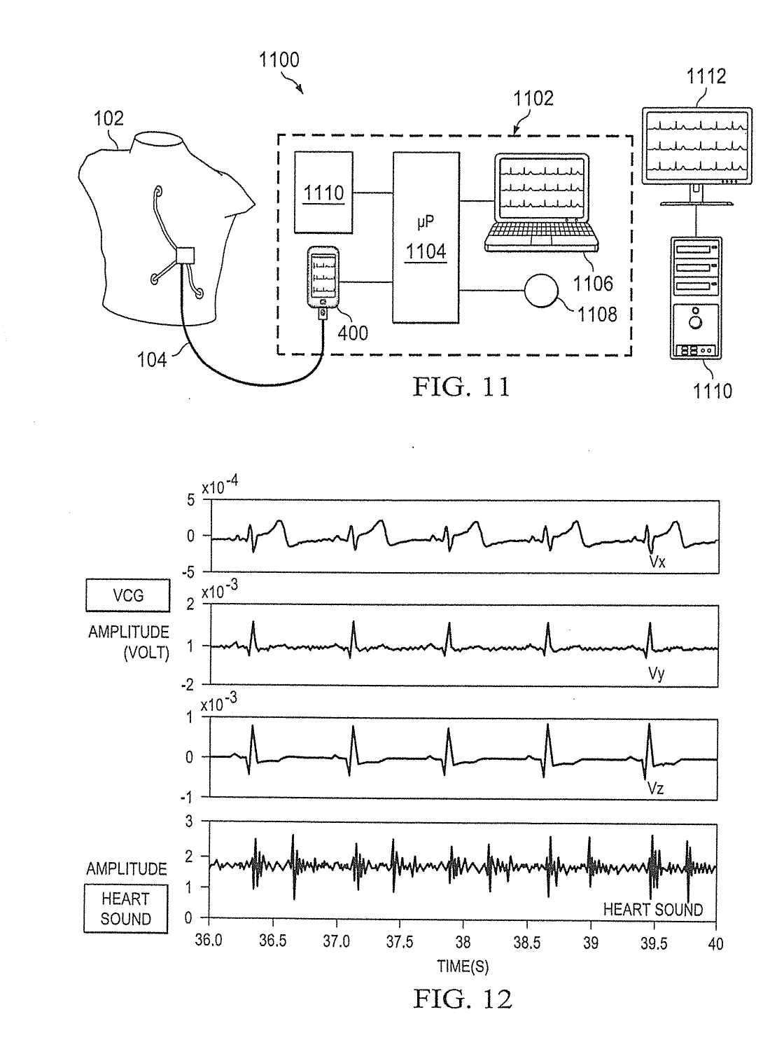

Referring now to FIG. 11, is a functional component diagram of a cardiac monitoring system 1100 according to the present disclosure is shown. Various embodiments of hardware configurations have been describe above. However, FIG. 11 provides a functional component view for purposes of illustration of how and where collected signals may be processed or handled. Here again, a patient torso 102 is shown with a plurality of leads 104 attached. Once again, a plurality of different ways to attach ECG and other leads to a patient are known to those of skill in the art. However, in the present disclosure, three leads 104 are utilized in a system 1100 capable of producing a data set or display corresponding to a traditional 12 lead ECG.

The three leads 104 may be passed to a monitoring unit 1102. The monitoring unit 1102 may be a standalone, dedicated device to collect and/or monitor data from the leads 104. In some embodiments, the device 1102 is portable or wearable. It may have a built in display device 1106, or it may simple collect or store data for transmission or display on a separate device. In the present embodiment, the leads 104 are passed to a signal conditioning circuit 400 that may operate as described with respect to FIG. 4 above. Amplification, filtering, offset or inversions needed to present a clear signal for processing may be handled by the circuit 400. The present disclosure is not meant to be limited by the analog signal conditioning aspects of the devices described herein.

A programmable microprocessor 1104 accepts the conditioned signals from the circuit 400. In some embodiments, the microprocessor 400 provides for A/D conversions of the signals from the circuit 400. In other embodiments, the circuit 400 may provide this functionality and provide fully digitized signals to the microprocessor 1104. In some embodiments, the microprocessor 1104 is general purpose programmable microprocessor as are known to those of skill in the art. The signals from the circuit 400 may be properly graphed and displayed on the integrated display 1106 (if provided). The microprocessor 1104 may have a memory 1110 associated therewith for storing the data collected. In some embodiments, the data stored corresponds to a 3 lead vector cardiogram. As described more fully below, the microprocessor 1104 can convert this information, as needed, into a 12 lead ECG for display on the display 1106. In some embodiments, the user is able to switch between vector cardiogram display and 12 lead ECG display as conditions warrant. This applies whether the data is being displayed in real time or being reviewed at a later time.

It is understood that the device 1102 may be based upon, or programmed into, a consumer electronic device such as a smart phone, tablet, or personal computer. It may also be a purpose-built device. The device 1102 may have a wireless communication module 1108 for providing data in real time, or for submitting archived data to an outside location. This may be a Bluetooth module as described but it may also be an 802.11 module or a device implementing another wireless protocol. FIG. 11 illustrates an outside computer 1110 that accepts wireless data from the patient device 1102. The outside computer 1110 may receive data in real time or may receive archived data. In some embodiments, events may be monitored by the patient device 1102 and then transmitted to the outside computer 1110 for further review and/or archival. It will also be appreciated that a number of wired connections as known in the art could be utilized. Data may also be transmitted via a network such as the Internet to allow for remote reception of monitored events by doctors or other medical professionals.

An important function provided by the systems of the present disclosure, is the ability to transform 3 lead vector cardiograms into 12 lead ECG signals without any statistically significant data loss. This capability is described herein and may be programmed into the microprocessor 1104 or other computationally enabled device of the present disclosure. In one embodiment, this may be described as a linear affine transformation between a 3-lead (Frank XYZ leads) vectorcardiogram signal and a 12-lead electrocardiogram signal.

For deriving the 12-lead ECG from the known Frank XYZ leads, randomly chosen recordings of healthy control individuals are used. From each recording, 2000 points were extracted from the mid portion of each signal, and all the selected recordings for training were concatenated to generate an input matrix of known lead values for a least squares fit. A linear regression fitting process was used to yield the affine transform matrix to derive 8 leads from the known 3 Frank (XYZ) VCG leads. In the present system, 8 leads are derived owing to the fact that in a 12 lead ECG, the remaining leads contain redundant data. An affine transform is commonly used for deriving multiple regression models. These models can compensate for constant bias and inconsistent baselines in the ECG signals.

A linear regression model assumes that every derived lead (here, the individual 8-lead values denoted as Y=[y₁, y₂, . . . , y₈]^{T}, where T denotes a transpose) can be obtained from a linear combination of the 3 Frank VCG values (here, the 3 leads are denoted as X=[x₁, x₂, x₃]), ie,

Y=AX+ε=a₀+a₁x₁+a₂x₂+a₃x₃+ε (1)

where a0, ai . . . an are the columns of the transform coefficients, and A is the transform matrix and c is the error. Thus, from knowing the input lead values, the corresponding coefficient vectors can be used to derive each of the 8 leads. We found that at least 25 recordings are needed to provide statistically consistent distribution of transform matrix values.

For deriving the three Frank XYZ leads from eight independent ECG leads (out of 12, because the augmented leads are calculated from leads I, II, and III), the fitting system becomes overdetermined, ie, the same XYZ lead values can be obtained from a given 12-lead values using completely different transform coefficients. This is because the eight leads carry redundant information. For example, leads II and III can have highly redundant information, and so do V5 and V6. This redundancy needs to be reduced to achieve consistent 8-lead to 3-lead transformation. Principal component analysis (PCA) is widely used in engineering to reduce such data redundancy. This technique was used in generating the present disclosure to determine a consistent transformation matrix to derive three Frank XYZ lead values.

The table below provides one example of a transformation matrix derived according to the present disclosure for HC (healthy control) and Myocardial Infarction (MI) patients:

In addition to the foregoing, the Dower matrix, as is known to those in art, may be utilized for transforming the 3 lead VCG data to the 12 lead ECG data.

TABLE 1
VCG to ECG transform coefficients (units mV/mV)

| Leads | X Lead | Y Lead | Z Lead |
|---|---|---|---|
| | HC | | |
| I | 0.5142 ± 0.0305 (0.632)* | −0.0582 ± 0.0390 (−0.235)* | −0.0948 ± 0.0084 (0.059)* |
| II | 0.2211 ± 0.0302 (0.235) | 0.9545 ± 0.0403 (1.066) | −0.0454 ± 0.0052 (−0.132)* |
| III | −0.2932 ± 0.0237 (−0.397)* | 1.0127 ± 0.0364 (1.301)* | 0.0494 ± 0.0099 (−0.191)* |
| aVR | −0.3676 ± 0.0279 (−0.434) | −0.4481 ± 0.0352 (−0.415) | 0.0701 ± 0.0049 (0.037)* |
| aVL | 0.4037 ± 0.0227 (0.515)* | −0.5354 ± 0.0319 (−0.768)* | −0.0721 ± 0.0088 (0.125)* |
| aVF | −0.0360 ± 0.0225 (−0.081) | 0.9836 ± 0.0331 (1.184) | 0.0020 ± 0.0067 (−0.162)* |
| V1 | −0.4500 ± 0.0455 (−0.515) | −0.1448 ± 0.0760 (0.157) | −0.8010 ± 0.0246 (−0.917)* |
| V2 | −0.1905 ± 0.0594 (0.044)* | −0.3183 ± 0.1211 (0.164) | −1.7516 ± 0.0347 (−0.139)* |
| V3 | 0.3532 ± 0.0343 (0.882)* | −0.0945 ± 0.0400 (0.098) | −1.6875 ± 0.0155 (−1.277)* |
| V4 | 1.0004 ± 0.0472 (1.213)* | 0.0569 ± 0.0530 (0.127) | −0.9643 ± 0.0186 (−0.601)* |
| V5 | 1.0996 ± 0.0360 (1.125) | 0.3009 ± 0.0453 (0.127)* | −0.2366 ± 0.0312 (−0.086)* |
| V6 | 0.8619 ± 0.0271 (0.831) | 0.2574 ± 0.0369 (0.076)* | 0.1077 ± 0.0182 (0.23)* |
| | MI | | |
| I | 0.7998 ± 0.0209 (0.632)* | −0.1600 ± 0.0303 (−0.235) | 0.0634 ± 0.0124 (0.059)* |
| II | 0.2647 ± 0.0116 (0.235) | 0.8977 ± 0.0286 (1.066) | −0.0285 ± 0.0044 (−0.132)* |
| III | −0.5351 ± 0.0188 (−0.397)* | 1.0576 ± 0.0268 (1.301)* | −0.0919 ± 0.0140 (−0.191)* |
| aVR | −0.5322 ± 0.0141 (−0.434)* | −0.3688 ± 0.0262 (−0.415) | −0.0175 ± 0.0061 (0.037)* |
| aVL | 0.6674 ± 0.0190 (0.515)* | −0.6088 ± 0.0248 (−0.768)* | 0.0777 ± 0.0130 (0.125)* |
| aVF | −0.1352 ± 0.0116 (−0.081)* | 0.9776 ± 0.0232 (1.184)* | −0.0602 ± 0.0083 (−0.162)* |
| V1 | −0.5325 ± 0.0196 (−0.515) | −0.3213 ± 0.0332 (0.157)* | −0.9793 ± 0.0341 (−0.917) |
| V2 | 0.0010 ± 0.0248 (0.044) | −0.6852 ± 0.0792 (0.164)* | −1.7674 ± 0.0522 (−0.1387)* |
| V3 | 0.5269 ± 0.0283 (0.88) | −0.3857 ± 0.0659 (0.098)* | −1.8725 ± 0.0484 (−1.277)* |
| V4 | 1.0550 ± 0.0330 (1.213)* | −0.1265 ± 0.0356 (0.127)* | −1.2897 ± 0.0490 (−0.601)* |
| V5 | 1.1306 ± 0.0195 (1.125) | 0.1941 ± 0.0353 (0.127) | −0.2893 ± 0.0191 (−0.086)* |
| V6 | 0.8176 ± 0.0146 (0.831) | 0.3113 ± 0.0336 (0.076) | 0.1049 ± 0.0074 (0.23) |

*denotes Dower values that do not fall within 3 × STD of the mean of the derived values.

The table shows coefficients of the affine matrix) with the corresponding Dower values shown in parentheses.

As previously described, the multi-sensor systems of the present disclosure (e.g., combining electrical and acoustic sensors) allows synchronous acquisition of cardiac system electrical and acoustic activity. Consequently, the time correlations between electrical and acoustic activities are preserved. This may results in more accurate capturing of cardiac activity (cardiovascular and cardiorespiratory dynamics). This aspect is demonstrated by showing a 2-5% improvement in the accuracy of respiration rate signals derived from combining heart sound and ECG signals than with the individual signals alone. The accuracy in the table below is estimated based on how well correlated the signals are with an actual measurement of respiration.

TABLE 2
Comparison among of respiration signals derived from ECG signal,
heart sound signals, and the combination of both signals.

| Test | | Correlation with Measured |
|---|---|---|
| Position | Derived source | Respiration signals |
| Supine | Heart sound and ECG | 0.89 |
| | ECG | 0.83 |
| | Heart sound | 0.815 |
| Upright | Heart sound and ECG | 0.87 |
| | ECG | 0.85 |
| | Heart sound | 0.78 |

The respiratory rate from ECG signals (EDR) techniques may be classified into four categories. The first category is based on estimating ECG-derived respiratory (EDR) signals using heart beat interval fluctuations, also referred to as respiratory sinus arrhythmia (RSA). The second category, the “amplitude method” is based on the fact that the impedance across the thoracic cavity changes during inspiration and expiration so that the amplitude of the ECG's QRS complex changes. Specifically, the EDR could be determined from the change in R-wave amplitude from the suitable lead ECG. The third category called the “area method” derives the EDR from the ratio of the area under the QRS complex in multiple lead ECG. The fourth, the angle of mean electrical axis (AMEA) method that estimates the EDR by finding the area of the QRS complex from any two lead ECGs, and then obtaining the AMEA from the arctangent of the ratio of these areas.

Although heretofore actual heart sound-based respiration derivation has not been widely known, body sounds at the neck around the carotid artery may be used to obtain respiratory rate signals. Auscultation has been used to collect respiration rate for the detection of breathing disorders. Another technique, called phonopneumography, is employed to more accurately count respiration rate. Acoustic sensors using a microphone detect analog breath sound signals which can be digitized and analyzed using computer algorithms have been used monitor respiration rate. Respiration signals can also be derived by having an acoustic sensor carrying a microphone placed on the trachea. Bioacoustics signals have used to obtain respiration signals

Improvement in the detection of cardiorespiratory disorders is demonstrated by showing that by combining information from derived respiration and ECG, the detection of a cardiorespiratory disorder such as an obstructive sleep apnea can be improved. In some embodiments, the VCG or ECG readings are correlated with the respiration input by correlating the R peak of the electrical reading with the Si signal (see FIG. 12).

Here we use recurrence quantification analysis and a variety of data mining techniques to extract important information from the signals to detect and state the severity of apnea conditions. Severity of apnea conditions is reported in terms of Apnea-Hypoapnea index (AHI).

As shown in the Table below, four types of models were used:

TABLE 3
Summary of models

| Model | Inputs |
|---|---|
| 0 (Conventional inputs) | Standard procedure |
| I | respiration rate |
| II | ECG Heart rate |
| III | respiration rate and Heart Rate |

The test results suggest that a datamining method called autoneural model provides the best performance when used with respiration and heart rate (from ECG) in terms of a low misclassification rate (11.94%) and high specificity (85.84%) and lift (2.7). Table 4, below, shows the average performance in the detection of cardiovascular disorder (sleep apnea) using conventional signal (nasal) and signals collected from wireless multi-sensor platform (ECG and respiration).

TABLE 4

| Model | Sensitivity | Specificity | Error (%) | Lift |
|---|---|---|---|---|
| 0 | 88.11% | 90.04% | 10.91% | 1.97 |
| I | 87.05% | 75.29% | 20.00% | 1.75 |
| II | 96.47% | 84.70% | 14.00% | 1.25 |
| III | 91.93% | 85.84% | 11.94% | 2.70 |

An excellent match (<10% deviation) with an evaluator's AHIs also suggests that the approach is robust for real-world OSA detection applications. Table 5 below shows calculated AHI (with % deviations w.r.t the benchmark Model 0 shown in parenthesis) from five representative datasets (A, B, C, D, E).

TABLE 5

| | | | | | | Av. % |
|---|---|---|---|---|---|---|
| Model | A | B | C | D | E | dev. |
| 0 | 22.5 | 8.5 | 13.7 | 15 | 27.4 | — |
| I | 24.3 (7.2) | 9.5 (10.5) | 18.7 (26.8) | 20 (25.0) | 34.4 (20.4) | 17.99 |
| II | 28.8 (21.7) | 7.3 (17.2) | 19.3 (29.3) | 19 (21.05) | 34.1 (19.6) | 21.78 |
| III | 25 (10.0) | 8.5 (0.0) | 14.7 (6.8) | 16 (6.2) | 30.4 (9.7) | 6.56 |

Heart sounds may be estimated based on ensemble averaging. To validate this technique, we have compared the waveform characteristics obtained using heart sounds with traditional ECG-derived respiration techniques as well as with the real-time respiration rate measuring from Vivo Metrics. Specifically, we compared the signals based on how well the respiration waveform, the peak to peak intervals, RSA and zero crossing intervals of the derived signals compare with those from the measured respiration signals.

As heart sounds are easily accessible to the doctor, there will be significant cost savings in terms of reduced technology (sensors and accessories) required to estimate respiration as compared to traditional heart sound derived respiration. Reducing/eliminating the number of sensors required will make the process of collecting data much easier for the doctor and painless for the subject.

Recurrence quantification analysis (RQA) is a method of nonlinear data analysis that quantifies the number and duration of recurrences of a dynamical system presented by its phase space trajectory using recurrence plots. In this study, the respiration rate signals contain dynamical information that is useful for detection of OSA. An equivalent state space (attractor) of respiration rate signals can be reconstructed from the delayed coordinates of the measurement.

The recurrence plots provide a convenient means to capture the topological relationships in the form of a color map. The salient RQA features extracted from the signals here are summarized as shown in Table 6.

TABLE 6

| ROA Features | Equations |
|---|---|
| Recurrence Rate | $RR ( ɛ ) = \frac{1}{{N}^{2}} \times {\sum }_{i , j = 1}^{N} {R}_{i , j} ( ɛ )$ |
| Determinism | $DET = \frac{{\sum }_{l = {l}_{min}}^{N} lP ( l )}{{\sum }_{l = 1}^{N} lP ( l )}$ |
| Mean diagonal line length | $L = \frac{{\sum }_{l = {l}_{min}}^{N} lP ( l )}{{\sum }_{l = {l}_{min}}^{N} lP ( l )}$ |
| Longest diagonal length | Lₘₐₓ = max({lᵢ}ᵢ₌₁^{N}^{ₗ}) |
| Entropy | $ENTR = - {\sum }_{l = {l}_{min}}^{N} p ( l ) ln P ( l )$ |
| Laminarity | $LAM = \frac{{\sum }_{v = {v}_{min}}^{N} vP ( v )}{{\sum }_{v = 1}^{N} vP ( v )}$ |
| Trapping time | $TT = \frac{{\sum }_{v = {v}_{min}}^{N} vP ( v )}{{\sum }_{v = {v}_{min}}^{N} vP ( v )}$ |
| Maximal length of vertical lines | Vₘₐₓ = max({vₗ}ₗ₌₁^{N}^{ᵥ}) |

In this research, five data mining techniques are used to uncover the underlying patterns of the cardio-respiratory system for detection of OSA: neural network, autoneural, regression, decision tree, and ensemble modeling techniques. In this research, the neural network uses one hidden layer consisting of three neurons. The weight function in each neuron is automatically optimized based on the patterns discovered from training data. The autoneural technique is used to automatically search for an optimal setting for the neural network model. A decision tree model ranks each RQA feature input from its contribution to the final output to the tree. Then, the output is selected based on the rules created by each leaf of the tree. The ensemble model can be used to combine other modeling methods, such as neural network and regression models, and then form the rules based on the prior models for a better final model solution. The performance of these models is assessed using four model selection criteria as shown in Table 7 below:

TABLE 7

| Model Selection | |
|---|---|
| Criteria | Description |
| Misclassification | Rate at which misclassification occurs in the |
| Rate | validation data |
| Lift | Model improvement provided by the model with |
| | respect to the baseline (random guess probability) |
| Sensitivity | Ratio of true positives to the sum of true positives |
| | and false negatives |
| Specificity | Ratio of true negatives to the sum of true |
| | negatives and false positives |

### REFERENCES

A. Sobron, et al., “Evaluation of Methods for Estimation of Respiratory Frequency from the Ecg,” in Computing in Cardiology, 2010, pp. 513-516.
B. F. Womack, “The Analysis of Respiratory Sinus Arrhythmia Using Spectral Analysis and Digital Filtering,” Biomedical Engineering, IEEE Transactions on, 399-409, 1971.
S. Dingab, et al., “Derivation of Respiratory Signal from Single-Channel Ecgs Based on Source Statistics,” 2004.
C. O'Brien and C. Heneghan, “A Comparison of Algorithms for Estimation of a Respiratory Signal from the Surface Electrocardiogram,” Computers in biology and medicine, vol. 37, 305-314, 2007.
L. A. Lipsitz, et al., “Heart Rate and Respiratory Rhythm Dynamics on Ascent to High Altitude,” British heart journal, vol. 74, 390-396, 1995.
S. P. Arunachalam and L. F. Brown, “Real-Time Estimation of the Ecg-Derived Respiration (Edr) Signal Using a New Algorithm for Baseline Wander Noise Removal,” in Engineering in Medicine and Biology Society, 2009. EMBC 2009. Annual International Conference of the IEEE, 2009, pp. 5681-5684.
G. B. Moody, et al., “Derivation of Respiratory Signals from Multi-Lead Ecgs,” Computers in cardiology, vol. 12, 113-116, 1985.
A. Travaglini, et al., “Respiratory Signal Derived from Eight-Lead Ecg,” in Computers in Cardiology 1998, 1998, pp. 65-68.
B. Mazzanti, et al., “Validation of an Ecg-Derived Respiration Monitoring Method,” in Computers in Cardiology, 2003, 2003, pp. 613-616.
K. Behbehani, et al., “An Investigation of the Mean Electrical Axis Angle and Respiration During Sleep,” in Engineering in Medicine and Biology, 2002. 24th Annual Conference and the Annual Fall Meeting of the Biomedical Engineering Society EMBS/BMES Conference, 2002. Proceedings of the Second Joint, 2002, pp. 1550-1551.
S. Leanderson, et al., “Estimation of the Respiratory Frequency Using Spatial Information in the Vcg,” Medical engineering & physics, vol. 25, 501-507, 2003.
H. Miwa and K. Sakai, “Development of Heart Rate and Respiration Rate Measurement System Using Body-Sound,” in Information Technology and Applications in Biomedicine, 2009. ITAB 2009. 9th International Conference on, 2009, pp. 1-4.
E. Kaniusas, et al., “Acoustical Signal Properties for Cardiac/Respiratory Activity and Apneas,” Biomedical Engineering, IEEE Transactions on, vol. 52, 1812-1822, 2005.
Y. Fu, et al., “Pulmonary Disease Management System with Distributed Wearable Sensors,” in Engineering in Medicine and Biology Society, 2009. EMBC 2009. Annual International Conference of the IEEE, 2009, pp. 773-776.
M. J. Banet, “Vital Signs Monitor Used for Conditioning a Patient's Response,” 2009.
G. A. Pressler, et al., “Detection of Respiratory Sounds at the External Ear,” Biomedical Engineering, IEEE Transactions on, vol. 51, 2089-2096, 2004.
D. G. Tinkelman, et al., “Analysis of Breath Sounds in Normal and Asthmatic Children and Adults Using Computer Digitized Airway Phonopneumography (Cdap),” Respiratory medicine, vol. 85, 125-131, 1991.
P. Hult, et al., “A Method for Respiration Monitoring by the Use of a Bioacoustic Signal,” 2000.
J. Werthammer, et al., “Apnea Monitoring by Acoustic Detection of Airflow,” Pediatrics, vol. 71, 53-55, 1983.
P. Corbishley and E. Rodriguez-Villegas, “Breathing Detection: Towards a Miniaturized, Wearable, Battery-Operated Monitoring System,” Biomedical Engineering, IEEE Transactions on, vol. 55, 196-204, 2008.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A system comprising:
a plurality of electrical detector leads for attaching to a patient;
a signal conditioning circuit that receives a plurality of signals from the detector leads and applies amplification, and high and low pass filtering to the received signals to produce conditioned analog signals;
an analog to digital converter than converts the conditioned analog signals to digital signals;
a microprocessor that applies a transformation to the digital signals to produce a set of transformed signals that are greater in number than the plurality of analog signals; and
a display device that selectively displays the digital signals and the transformed signals.

2. The system of claim 1, further comprising a memory associated with the microprocessor for storing the digital signals.

3. The system of claim 1, further comprising a wireless communication chip that transmits the digital signals to a recipient device.

4. The system of claim 1, wherein the microprocessor computes heart rate variability for display on the display device.

5. The system of claim 1, wherein the plurality of detector leads correspond to a 3-lead vectorcardiogram.

6. The system of claim 5, wherein the digital signals correspond to a 12-lead electrocardiogram.

7. The system of claim 6, further comprising a microphone that captures heart sounds, wherein the microprocessor correlates the electrocardiogram signals to the heart sounds to determine respiration events.

8. The system of claim 6, wherein the microprocessor calculates energies of 3 separate vectorcardiogram leads for display on the display device.

9. A system comprising
a set of vectorcardiogram leads for attaching to a patient;
a signal conditioning circuit that receives a plurality of analog signals from the plurality of vectorcardiogram leads;
an analog to digital converter that transforms the conditioned analog signals into digital vectorcardiogram signals;
a processor that transforms the digital vectorcardiogram signals into electrocardiogram signals.

10. The system of claim 9, wherein the signal conditioning circuit and the processor are packaged into a portable electronic device.

11. The system of claim 9, further comprising a memory that stores the digital signals.

12. The system of claim 11, wherein the vectorcardiogram is a 3 lead vectorcardiogram and the electrocardiogram is a 12 lead electrocardiogram.

13. The system of claim 12, wherein the portable electronic device further comprises a display that selectively displays a vectorcardiogram and an electrocardiogram.

14. The system of claim 12, further comprising a wireless transmitter that delivers at least the 3 lead vectorcardiogram to another device.

15. The system of claim 14, wherein the microprocessor performs an affine transform of the 3 lead vectorcardiogram to produce the 12 lead electrocardiogram.

16. The system of claim 11, further comprising a microphone for detecting heart sounds, wherein the processor correlates the heart sounds to the ECG signal.

17. A method comprising:
attaching a portable device to a patient that obtains 3 lead vectorcardiogram reading from a patient;
transforming the vectorcardiogram in the portable device to a 12 lead electrocardiogram; and
displaying the 12 lead electrocardiogram.

18. The method of claim 17, further comprising transforming the vectorcardiogram in the portable device to a 12 lead electrocardiogram by performing an affine transform.

19. The method of claim 17, further comprising displaying the 12 lead electrocardiogram.

20. The method of claim 17, further comprising wirelessly transmitting the vectorcardiogram.
